(19) United States
(12) Patent Application Publication
(10) Pub. No.: US 2025/0255817 A1
(43) Pub. Date: Aug. 14, 2025

(54) FORMULATIONS COMPOSED OF CATIONIC LIPIDS AND POLY(LACTIC-CO-GLYCOLIC ACID) FOR THE DELIVERY OF POLYNUCLEOTIDES INTO CELLS

(71) Applicant: Evonik Operations GmbH, Essen (DE)

(72) Inventors: Philipp Heller, Mainz (DE); Alexander Bernhardt, Nieder-Olm (DE); Anne Benedikt, Frankfurt (DE); Hans Bär, Michelstadt (DE); Norbert Windhab, Hofheim (DE)

(73) Assignee: Evonik Operations GmbH, Essen (DE)

(21) Appl. No.: 18/850,154

(22) PCT Filed: Mar. 27, 2023
(86) PCT No.: PCT/EP2023/057752

§ 371 Date: Sep. 24, 2024

(30) Foreign Application Priority Data
Mar. 30, 2022 (EP) 22165385.0

(51) Int. Cl. A61K 9/16; A61K 31/7088

(52) U.S. Cl. CPC A61K 9/1647; A61K 31/7088

(57) ABSTRACT

A nanoprecipitation or nanoemulsion method forms a polynucleotide delivery particle, wherein the polynucleotide delivery particle contains at least one poly(lactic-co-glycolide), at least one cationic surfactant, at least one polynucleotide, and optionally at least one additive, wherein the poly(lactic-co-glycolide) has a weight average molecular weight Mw of 1000 to 9500 g/mol measured via gel permeation chromatography using polystyrene standards and chloroform. The polynucleotide delivery particle as an additional component in an oral drug delivery composition or a parenteral drug delivery composition supports the beneficial characteristics of the application as a medicament.

### FIELD OF THE INVENTION

The present invention refers to a polynucleotide delivery particle, comprising
a) at least one poly(lactic-co-glycolide);
b) at least one cationic surfactant;
c) at least one polynucleotide; and
d) optionally at least one additive; wherein
   the poly(lactic-co-glycolide) has a weight average molecular weight Mw of 1000 to 9500 g/mol measured via gel permeation chromatography using polystyrene standards and chloroform. Furthermore, the present invention pertains to a method of forming the polynucleotide delivery particle according to the present invention, wherein the particle is formed by a nanoprecipitation or nanoemulsion method. Moreover, the present invention refers to an oral drug delivery composition or a parenteral drug delivery composition comprising at least one polynucleotide delivery particle according to the present invention as well as their use as a medicament.

### BACKGROUND

Polynucleotide-based drugs are a novel class of therapeutics which emerged during the last 10 to 20 years. They hold great promise giving access to new treatment options for cancer therapy and vaccination as well as for previously undruggable diseases.

The main hurdle for a successful application of polynucleotide therapeutics is their delivery to the site of action, i.e., the cytoplasm or the cell nucleus. Polynucleotides are large biomolecules, which in general are prone to chemical and enzymatic degradation and do not readily enter cells. Thus, a suitable formulation must provide protection against any hazardous environment, which a polynucleotide drug will inevitably encounter upon local, systemic, or oral application. Additionally, a formulation should mediate the uptake of the polynucleotide drug into the target cell and eventually facilitate its release from endosomal compartments into the cytoplasm.

Of the many non-viral formulations known in the art, currently the so-called lipid nanoparticles (LNP) are the most advanced platform. Consequently, LNPs constitute the delivery vehicles in the first generation of commercially approved siRNA and mRNA-based drugs. LNPs are composed of up to four different types of surfactants (lipids) (a cationic/ionizable surfactant, a PEG surfactant, cholesterol, and phospholipid) which renders the formulation rather complex and expensive, especially with regard to the supply of raw materials. Moreover, the mandatory use of PEG surfactants raises concerns about possible immunogenic reactions which might be triggered due to the presence of anti-PEG antibodies in a subset of the population.

Because of their excellent biocompatibility, nano- and microparticles formed of poly(lactic-co-glycolic acid) (PLGA) are another widely used drug delivery platform. However, due to its charge-neutral and hydrophobic character PLGA itself is a rather unsuitable material for the encapsulation of hydrophilic, charged macromolecules as in the case of polynucleotides. In order to increase association with polynucleotides, the combination of PLGA with positively charged excipients, e.g. calcium phosphate, has been proposed as a workaround. Still, these emulsion-based strategies suffer from complicated multistep protocols, poor encapsulation efficiency and large particle sizes.

Helper surfactants (lipids) (cholesterol, phospholipid, PEG lipid) are basic requirements for the formation of LNPs, as the cationic/ionizable lipid and polynucleotide alone cannot co-assemble into efficient nanoparticles. Thus, the object of the present invention was the provision of PLGA based polynucleotide delivery particles, which can overcome one or more of the above-mentioned disadvantages.

In this regard, the inventors of the present invention surprisingly found that a polynucleotide can be entrapped into PLGA particles using a simple mixing protocol without the need for additional surfactants or helper lipids when a cationic lipid is applied as the positively charged excipient. Moreover, improved cell transfection can be obtained when the poly(lactic-co-glycolide) has a weight average molecular weight Mw of 1000 to 9500 g/mol.

Furthermore, the absence of PEG as required in LNPs allows the coating of the lipid/PLGA particles with other materials such as cell-penetrating peptides (e.g., human lactoferrin protein or fragments thereof) to adjust particle surface properties and increase functionality.

### SUMMARY OF THE INVENTION

In a first aspect the present invention refers to a polynucleotide delivery particle, comprising or consisting of
a) at least one poly(lactic-co-glycolide);
b) at least one cationic surfactant;
c) at least one polynucleotide; and
d) optionally at least one additive; wherein
   the poly(lactic-co-glycolide) has a weight average molecular weight Mw of 1000 to 9500 g/mol, preferably 2000 to 6800 g/mol, more preferably 4000 to 6800 g/mol, most preferably 6000 to 6800 g/mol, measured via gel permeation chromatography using polystyrene standards and chloroform.

In a second aspect the present invention pertains to a method of forming the polynucleotide delivery particle according to the present invention, wherein the particle is formed by a nanoprecipitation or nanoemulsion method.

In a third aspect the present invention refers to an oral drug delivery composition comprising at least one polynucleotide delivery particle according to the present invention.

In a fourth aspect the present invention pertains to a parenteral drug delivery composition comprising at least one polynucleotide delivery particle according to the present invention.

In a fifth aspect the present invention refers to an oral drug delivery composition according to the present invention or a parenteral drug delivery composition according to the present invention for use as a medicament.

### DESCRIPTION OF THE FIGURES

FIG. 1: Agarose gel electrophoresis of free mRNA and different DODMA:PLGA based particle samples. 1 μg of mRNA or the equivalent amount of particles were applied per well.

FIG. 2: Agarose gel electrophoresis of free mRNA and different DOTMA:PLGA based particle samples. 1 μg of mRNA or the equivalent amount of particles were applied per well.

FIG. 3: Transfection efficiency of different DODMA:PLGA based particle samples in Hela cells after 24 hours of incubation. 100 ng of mRNA per well were applied for each condition.

FIG. 4: Transfection efficiency of different DOTMA:PLGA based particle samples in Hela cells after 24 hours of incubation. 100 ng of mRNA per well were applied for each condition.

FIG. 5: Transfection efficiency of different DOTMA:PLGA based particle samples in Hela cells after different incubation times. 100 ng of mRNA per well were applied for each condition.

FIG. 6: Transfection efficiency of DOTMA:PLGA particles coated with different amounts of hLFF in Caco-2 cells after 24 hours of incubation. 100 ng of mRNA per well were applied for each condition.

FIG. 7: Release kinetics of DOTMA:PLGA particles after dissolution assay in 0.1 N HCl (0-120 minutes) and phosphate buffer pH 6.8 (120-180 minutes) as obtained by Ribogreen assay (representative from n=2).

FIG. 8: Transfection efficiency of DOTMA:PLGA particle samples in Hela cells after different pre-treatments and 24 hours of incubation. 100 ng of mRNA per well were applied for each condition.

### DETAILED DESCRIPTION

In one aspect the present invention refers to a polynucleotide delivery particle, comprising or consisting of
a) at least one poly(lactic-co-glycolide) (referred to as PLGA as well);
b) at least one cationic surfactant;
c) at least one polynucleotide; and
d) optionally at least one additive; wherein
   the poly(lactic-co-glycolide) has a weight average molecular weight Mw of 1000 to 9500 g/mol, preferably 2000 to 6800 g/mol, more preferably 4000 to 6800 g/mol, most preferably 6000 to 6800 g/mol, measured via gel permeation chromatography using polystyrene standards and chloroform.

The polynucleotide delivery particles can be nanoparticles or microparticles. In certain embodiments, the particles have a D(v,0.5) value that is between 50 and 500 nanometers and/or a z-average particle size of 1 to 1000 nanometers, preferably 20 to 500 nanometers, more preferably 20 to 200 nanometers.

The term “particle” as used herein, preferably refers to a particle having a size less than 10 μm (10,000 nm), for example, ranging from about 1 nm to 25 nm, to 50 nm, to 100 nm, to 250 nm, to 500 nm, to 1000 nm (1 μm), to 2,500 nm (2.5 μm), to 5,000 nm (5 μm), or to 10,000 nm (10 μm). In some embodiments, dry particles may exist in aggregates that are greater than 10,000 nm in diameter, but which disperse into particle sizes less than 10,000 nm upon addition of an aqueous fluid and mixing using techniques such as vortexing. In some embodiments, the particles described herein can be generally spherical. In some embodiments, the particles described herein can be of irregular geometry. The particles within the compositions of the present invention typically have a size distribution in aqueous fluid, wherein the z-average and/or the D(v,0.5) value is less than 5,000 nm, for example, ranging from 5,000 nm to 2,500 nm, to 1,000 nm, to 500 nm, to 250 nm, to 100 nm, to 50 nm, or to 1 nm.

As used herein “nanoparticles” are particles that have a size distribution in aqueous fluid in which the z-average ranges from 1 nm to 500 nm. As used herein “microparticles” are particles that have a size distribution in aqueous fluid in which the D(v,0.5) ranges from 500 nm to 5000 nm.

Particle size can be determined (measured) using methods available in the art. For example, particle size can be determined using photon correlation spectroscopy, dynamic light scattering or quasi-elastic light scattering. These methods are based on the correlation of particle size with diffusion properties of particles obtained from Brownian motion measurements. Brownian motion is the random movement of the particles due to bombardment by the solvent molecules that surround the particles. The larger the particle, the more slowly the Brownian motion will be. Velocity is defined by the translational diffusion coefficient (D). The value measured refers to how a particle moves within a liquid (hydrodynamic diameter). The diameter that is obtained is the diameter of a sphere that has the same translational diffusion coefficient as the particle.

Particle size can also be determined using static light scattering, which measures the intensity of light scattered by particles in a solution at a single time. Static light scattering measures light intensity as a function of scattering angle and solute concentration. Particles passing through a light source, for example, a laser beam, scatter light at an angle that is inversely proportional to their size. Large particles generate a diffraction pattern at low scattering angles with high intensity, whereas small particles give rise to wide angle low intensity signals. Particle size distributions can be calculated if the intensity of light scattered from a sample is measured as a function of angle. The angular information is compared with a scattering model (e.g., Mie theory) in order to calculate the size distribution.

Generally, particle size is determined at room temperature and involves multiple analyses of the sample in question (e.g., at least 3 repeat measurements on the same sample) to yield an average value for the particle diameter.

The values are preferably determined via dynamic light scattering, more preferably according to DIN ISO 22412:2018-09.

The polynucleotide delivery particles can have a polydispersity index of 0.01 to 0.5, preferably measured via dynamic light scattering, more preferably according to DIN ISO 22412:2018-09.

In one embodiment the polynucleotide delivery particle according to the invention has a N/P ratio of the cationic surfactant to the polynucleotide from 1:1 to 50:1, preferably 5:1 to 20:1, more preferably the ratio is 8.

In one embodiment the polynucleotide delivery particle according to the invention has a weight ratio of poly(lactic-co-glycolide) to the polynucleotide is from 1 to 200 or 2 to 150 or 5 to 100.

The polynucleotide delivery particle comprises at least one poly(lactic-co-glycolide) having a weight average molecular weight Mw of 1000 to 9500 g/mol, preferably 2000 to 6800 g/mol, more preferably 4000 to 6800 g/mol, most preferably 6000 to 6800 g/mol, measured via gel permeation chromatography using polystyrene standards and chloroform.

In one embodiment the at least one poly(lactic-co-glycolide) has a number average molecular weight Mn of 1000 to 3000 g/mol, preferably 2000 to 2800 g/mol measured via gel permeation chromatography using polystyrene standards and chloroform.

In one embodiment the at least one poly(lactic-co-glycolide) has a lactide to glycolide molar ratio ranging from 40:60 to 60:40, preferably 50:50.

In one embodiment the at least one poly(lactic-co-glycolide) has an inherent viscosity of 0.05 to 0.25 dl/g, preferably 0.08 to 0.16, measured via viscometry.

In one embodiment the at least one poly(lactic-co-glycolide) has an acid number of 20 to 30, preferably 22.5, mg KOH/g, preferably measured according to DIN EN 14104:2021-04.

In one embodiment the at least one poly(lactic-co-glycolide) is present in 0.26 to 98.5 wt.-%, based on the total weight of the polynucleotide delivery particle.

Suitable poly(lactic-co-glycolide) polymers are for example commercially available under the tradename RESOMER® from Evonik Industries AG, like RESOMER® RG 501 H or RESOMER® Condensate RG polymers.

The particles comprise at least one cationic surfactant.

The term “surfactant” comes from the phrase “surface active agent”. Surfactants accumulate at interfaces (e.g., at liquid-liquid, liquid-solid and/or liquid-gas interfaces) and change the properties of that interface. As used herein, surfactants include detergents, dispersing agents, suspending agents, emulsion stabilizers, neutral lipids, ionizable lipids, cationic lipids, and anionic lipids.

Cationic surfactants are provided to impart charge to the particles.

In one embodiment the at least one cationic surfactant is selected from salts of 1,2-di-O-octadecenyl-3-trimethylammonium propane, 1,2-dioleoyl-3-trimethylammonium-propane, N1-[2-((1S)-1-[(3-aminopropyl)amino]-4-[di(3-amino-propyl)amino]butylcarboxamido)ethyl]-3,4-di[oleyloxy]-benzamide, N4-cholesteryl-spermine, 3ß-[N—(N′,N′-dimethylaminoethane)-carbamoyl]cholesterol, O,O′-ditetradecanoyl-N-(α-trimethylammonioacetyl) diethanolamine, 1,2-dilauroyl-sn-glycero-3-ethylphosphocholine, 1,2-dimyristoyl-sn-glycero-3-ethylphosphocholine, 1,2-dipalmitoyl-sn-glycero-3-ethylphosphocholine, 1,2-distearoyl-sn-glycero-3-ethylphosphocholine, 1,2-dioleoyl-sn-glycero-3-ethylphosphocholine, 1-palmitoyl-2-oleoyl-sn-glycero-3-ethylphosphocholine, 1,2-dimyristoleoyl-sn-glycero-3-ethylphosphocholine, dimethyldioctadecylammonium, 1,2-dimyristoyl-3-trimethylammonium-propane, 1,2-dipalmitoyl-3-trimethylammonium-propane, 1,2-stearoyl-3-trimethylammonium-propane, N-(4-carboxybenzyl)-N,N-dimethyl-2,3-bis(oleoyloxy)propan-1-aminium and 3ß-[N—(N′,N′-dimethylaminoethane)-carbamoyl]cholesterol.

In one embodiment the at least one cationic surfactant is a 1,2-di-O-octadecenyl-3-trimethylammonium propane salt, preferably 1,2-di-O-octadecenyl-3-trimethylammonium propane chloride salt.

In one embodiment the at least one cationic surfactant is present in 0.85 to 98 wt.-%, based on the total weight of the polynucleotide delivery particle.

In this regard, various salt forms of the preceding cationic surfactants may be provided including halide and hydrohalide salts such as chloride, bromide, iodide, and hydrochloride. Where a particular salt is listed (e.g., chloride), it is to be understood that other salts (e.g., bromide, iodide, etc.) may be employed as well. In one embodiment preferred salts of the cationic surfactants are chloride and bromide, more preferred are chloride salts.

The particles of the present invention comprise at least one polynucleotide.

As used herein, the term “polynucleotide” means a homopolymer or heteropolymer of at least 2 nucleotide units (also referred to herein as “nucleotides”). Nucleotides forming polynucleotides as defined herein include naturally occurring nucleotides, such as ribonucleotides and deoxyribonucleotides, as well as equivalents, derivatives, variants, and analogues of naturally occurring nucleotides.

In one embodiment the polynucleotide consists of 10 to 15000 nucleotides, preferably 20 to 5000 nucleotides, more preferably 500 to 4500 nucleotides.

A polynucleotide may be in either single-stranded form or multi-stranded form (e.g., double-stranded, triple-stranded, etc.). A polynucleotide may be in linear form or non-linear form (e.g., comprising circular, branched, etc. elements). A polynucleotide may be natural, synthetic or a combination of both.

A polynucleotide may be capable of self-replication when introduced into a host cell. Examples of polynucleotides thus include self-replicating RNAs and DNAs and, for instance, selected from replicons, plasmids, cosmids, phagemids, transposons, viral vectors, artificial chromosomes (e.g., bacterial, yeast, etc.) as well as other self-replicating species.

Polynucleotides include those that express antigenic polypeptides in a host cell (e.g., polynucleotide-containing antigens). Polynucleotides include self-replicating polynucleotides within which natural or synthetic sequences derived from eucaryotic or prokaryotic organisms (e.g., genomic DNA sequences, genomic RNA sequences, cDNA sequences, etc.) have been inserted. Specific examples of self-replicating polynucleotides include RNA vector constructs and DNA vector constructs, among others. Sequences that may be expressed include native sequences and modifications, such as deletions, additions, and substitutions (generally conservative in nature), to native sequences, among others.

These modifications may be deliberate, as through site-directed mutagenesis, or may be accidental, such as through mutations of hosts that produce antigens.

In one embodiment the at least one polynucleotide is selected from single-stranded or multi-stranded polynucleotides, preferably from an artificial messenger RNA (mRNA), chemically modified or unmodified mRNA comprising at least one coding sequence, self-replicating RNA, circular RNA, viral RNA, and replicon RNA; from linear DNA, plasmid DNA (pDNA), minicircle DNA, doggybone DNA (dbDNA); from small interfering RNA (siRNA), micro RNA (miRNA), guide RNA, small activating RNA (saRNA), antisense oligonucleotides (ASO); or any combination thereof, most preferably an mRNA.

In one embodiment the at least one polynucleotide is comprised in 0.1 to 50 wt.-%, preferably 0.2 to 40 wt.-%, more preferably 0.3 to 35 wt.-%, based on the total weight of the polynucleotide delivery particle.

The polynucleotide delivery particle according to the present invention can comprise at least one additive.

In one embodiment the weight ratio of the at least one additive to the at least one polynucleotide ranges from 0.01 to 50; or 0.01 to 30; or 0.01 to 10; or 0.01 to 5; or 0.01 to 2; or 0.01 to 1; or 0.01 to 0.1.

In one embodiment the at least one additive, preferably cell penetrating peptide, more preferably human lactoferrin protein or a fragment thereof, is present in 0.01 to 88 wt.-%, based on the total weight of the polynucleotide delivery particle.

Any known additive in the field is suitable, if it is pharmaceutically acceptable.

By “pharmaceutically acceptable” is meant a material which is not biologically or otherwise undesirable, i.e., the material may be administered to an individual without causing any excessively undesirable biological effects in the individual or interacting in an excessively deleterious manner with any of the components of the composition in which it is contained.

The term “additive” for example includes buffers such as phosphate, acetate, citrate, 4-(2-hydroxyethyl)-1-piperazineethanesulfonic acid, and other organic compounds; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; carbohydrates including monosaccharides, disaccharides, and other glucose, mannose, or dextrins; chelating agents such as EDTA; sugars such as sucrose, mannitol, trehalose or sorbitol; salt-forming counter-ions such as sodium; metal complexes (e.g., Zn-protein complexes), vehicles, binders, disintegrants, immunological adjuvants like a cell penetrating peptide, for example human lactoferrin protein or a fragment thereof, Tat, Ant, Rev, FHV, HSV-1 protein VP22, C6, C6M1, PF20, NAP, POD, polyarginine, polylysine, PTD-5, Transportan, MAP, TP10, Pep-7, Azurin p18, Azurin p28, hCT18-32, Bac 7, CTP, K5-FGF, HAP-1, 293P-1, KALA, GALA, LAH4-L1, Melittin, Penetratin, EB1, MPG, CADY, Pep4, preferably a human lactoferrin protein or a fragment thereof, fillers (diluents), lubricants, glidants (flow enhancers), compression aids, colors, cryoprotective agents, sweeteners, suspending/dispersing agents, film formers/coatings, flavors, printing inks, non-ionic surfactants, ionizable surfactants, lipids such as cholesterol, phospholipids, sphingolipids, ceramides, fatty acids; lipids linked to a hydrophilic polymer.

In one embodiment the polynucleotide delivery particle comprises at least one additive selected from buffers; cryoprotective agents; ionizable surfactants; non-ionic surfactants; lipids such as cholesterol, phospholipids, sphingolipids, ceramides, fatty acids; lipids linked to a hydrophilic polymer.

In one embodiment the polynucleotide delivery particle solution comprises at least one buffer, preferably in an amount of 0.1 mM to 1000 mM, based on the total volume of the polynucleotide delivery particle solution. The at least one buffer is preferably selected from PBS, phosphate buffer, acetate buffer, and (4-(2-hydroxyethyl)-1-piperazineethanesulfonic acid).

In one embodiment the polynucleotide delivery particle comprises less than 0.5 wt.-% of non-ionic surfactant, preferably less than 0.1 wt.-% of non-ionic surfactant, more preferably no non-ionic surfactant, based on the total weight of the polynucleotide delivery particle.

Suitable non-ionic surfactants vary widely, and numerous examples are described below. In certain preferred embodiments, the non-ionic surfactant is selected from poly(vinyl alcohol), polysorbate (e.g., polysorbate 20, polysorbate 80) and poloxamers.

In one embodiment the polynucleotide delivery particle further comprises at least one ionizable surfactant, preferably in a N/P ratio of the ionizable surfactant to the polynucleotide from 1 to 50. The at least one ionizable surfactant is preferably selected from salts of 1,2-distearoyl-3-dimethylammonium-propane, 1,2-dipalmitoyl-3-dimethylammonium-propane, 1,2-dimyristoyl-3-dimethylammonium-propane, 1,2-dioleoyl-3-dimethylammonium-propane, 1,2-dioleyloxy-3-dimethylaminopropane, (6Z,9Z,28Z,31Z)-heptatriacont-6,9,28,31-tetraene-19-yl 4-(dimethylamino) butanoate, 9-Heptadecanyl 8-{(2-hydroxyethyl)[6-oxo-6-(undecyloxy)hexyl]amino}octanoate, N,N-dimethyl-2,2-di-(9Z,12Z)-9,12-octadecadien-1-yl-1,3-dioxolane-4-ethanamine, [(4-hydroxybutyl)azanediyl]di(hexane-6,1-diyl) bis(2-hexyldecanoate).

In certain embodiments, particles according to the present invention can comprise immunological adjuvants. Examples of immunological adjuvants include E. coli heat-labile toxins, alum, liposaccharide phosphate compounds, liposaccharide phosphate mimetics, monophosphoryl lipid A analogues, small molecule immune potentiators, muramyl tripeptide phosphatidylethanolamine, and tocopherols.

The immunological adjuvants may be, for example, associated with the surface of the particles (e.g., adsorbed or otherwise bound), entrapped within the particles, or both. Immunological adjuvants increase or diversify the immune response to an antigen. Hence, immunological adjuvants are compounds that are capable of potentiating an immune response to antigens. Immunological adjuvants can potentiate humoral and/or cellular immunity.

In one embodiment the particle has an outer coating layer or at least one additive adsorbed to the surface of the particle, preferably a cell penetrating peptide, more preferably human lactoferrin protein or a fragment thereof. The human lactoferrin protein or fragment thereof according to the invention is for example described in WO 2007076904 A1 and is as well as its manufacturing process incorporated by reference.

In one embodiment the weight ratio of the cell penetrating peptide, preferably human lactoferrin protein or fragment thereof, to the at least one polynucleotide ranges from 0.01 to 50, preferably 0.1 to 30.

As defined herein, “carbohydrates” include monosaccharides, oligosaccharides and polysaccharides, as well as substances derived from monosaccharides, for example, by reduction (e.g., alditols), by oxidation of one or more terminal groups to carboxylic acids (e.g., glucuronic acid), or by replacement of one or more hydroxy group(s) by a hydrogen atom or an amino group (e.g., beta-D-glucosamine and beta-D-galactosamine).

As defined herein, a “monosaccharide” is a polyhydric alcohol, i.e., an alcohol that further comprises either an aldehyde group (in which case the monosaccharide is an aldose) or a keto group (in which case the monosaccharide is a ketose). Monosaccharides typically contain from 3 to 10 carbons. Moreover, monosaccharides commonly have the empirical formula CₙH₂ₙOₙ where n is an integer of three or greater, typically 3-10. Examples of 3-6 carbon aldoses include glyceraldehyde, erythrose, threose, ribose, 2-deoxyribose, arabinose, xylose, lyxose, allose, altrose, glucose, mannose, gulose, idose, galactose, and talose.

Examples of 3-6 carbon ketoses include dihydroxyacetone, erythrulose, ribulose, xylulose, psicose, fructose, sorbose, and tagatose. Naturally occurring monosaccharides are normally found in the D-isomer form, as opposed to the L-form.

An “oligosaccharide” refers to a relatively short monosaccharide polymer, i.e., one containing from 2 to 30 monosaccharide units. A “polysaccharide” is a monosaccharide polymer that is beyond oligosaccharide length (i.e., one containing more than 30 monosaccharide units). Moreover, as used herein, the term “polysaccharide” also refers to a monosaccharide polymer that contains two or more linked monosaccharides. To avoid ambiguity, the second definition is to be applied at all times, unless there are explicit indications to the contrary. The term “polysaccharide” also includes polysaccharide derivatives, such as amino-functionalized and carboxyl-functionalized polysaccharide derivatives, among many others. Monosaccharides are typically linked by glycosidic linkages. Specific examples include disaccharides (such as sucrose, lactose, trehalose, maltose, gentiobiose and cellobiose), trisaccharides (such as raffinose), tetrasaccharides (such as stachyose), and pentasaccharides (such as verbascose).

As used herein the term “saccharide” encompasses monosaccharides, oligosaccharides and polysaccharides. A “saccharide-containing species” is a molecule, at least a portion of which is a saccharide. Examples include saccharide cryoprotective agents, saccharide antigens, antigens comprising saccharides conjugated to carrier peptides, and so forth. A “polysaccharide-containing species” is a molecule, at least a portion of which is a polysaccharide.

As used herein, a “cryoprotective agent” is an agent that protects a composition from experiencing adverse effects upon freezing and thawing. For example, in the present invention, cryoprotective agents such as polyols and/or carbohydrates, among others, may be added to prevent substantial particle agglomeration from occurring when the lyophilized compositions of the invention are resuspended.

Various methods may be employed to produce particles according to the invention. For example, nanoprecipitation, i.e., mixing of an aqueous phase containing the polynucleotide with a water-miscible organic phase containing the excipients and additives or nanoemulsion, i.e., mixing of an aqueous phase containing the polynucleotide with a non-water-miscible organic phase containing the excipients and additives, can be used.

In some embodiments the particles may be formed using an oil-in-water (o/w) or water-in-oil-in-water (w/o/w) solvent evaporation process or using a nanoemulsion method.

The w/o/w solvent evaporation process is described, for example, in O'Hagan et al, Vaccine (1993) 11:965-969, Jeffery et al, Pharm. Res. (1993) 10:362, and WO 00/06123 A1. PLGA and a cationic surfactant (e.g., selected from those listed above, among others) are dissolved in one or more organic solvent(s) to form an organic solution. The solvent or solvent mixture may comprise one or more organic solvent(s), for example, selected from dichloromethane (DCM), ethyl acetate (EtOAc), chloroform, benzyl alcohol, diethyl carbonate (DMC), dimethyl sulfoxide (DMSO), methanol, propylene carbonate, isopropyl acetate, methyl acetate, methyl ethyl ketone, butyl lactate and isovaleric acid or any mixture thereof. A preferred solvent or solvent mixture may comprise EtOAc, DCM, EtOAc and DMSO or DCM and DMSO. The organic solution is then combined with a first volume of aqueous solution containing at least one polynucleotide and emulsified to form a water-in-oil emulsion. The aqueous solution can be, for example, deionized water, normal saline, a buffered solution, for phosphate-buffered saline (PBS) or a sodium citrate/example, ethylenediaminetetraacetic acid (sodium citrate/ETDA) buffer solution, among others. Typically, the volume ratio of organic solution to aqueous solution ranges from about 2:1 to about 20:1, more typically about 10:1. Emulsification is conducted using any equipment appropriate for this task. The most common approaches involve simple mechanical stirring, sonication, high shear mixing (HSM), high pressure homogenization (HPH), and microfluidics or millifluidics such as T or Y mixing.

A volume of the water-in-oil emulsion is then combined with a larger second volume of an aqueous solution, which may contain an emulsion stabilizing agent, for instance, an uncharged surfactant (e.g., PVA (polyvinyl alcohol), povidone (also known as polyvinylpyrrolidone or PVP), sorbitan esters, polysorbates, or poloxamers, among others) or an anionic surfactant or a cationic surfactant (e.g., selected from those listed above, among others). The volume ratio of aqueous solution to the water-in-oil emulsion typically ranges from about 2:1 to 20:1, more typically about 4:1. This mixture is then homogenized to produce a stable w/o/w double emulsion. Organic solvents are then evaporated to yield particles.

The nanoprecipitation method, also referred to as the solvent displacement method, is another example of a suitable method for forming particles for use in the invention. See, e.g., EP 0274961 B1 entitled “Process for the preparation of dispersible colloidal systems of a substance in the form of nanocapsules” Devissaguet et al, U.S. Pat. No. 5,049,322 by the same title, Fessi et al, U.S. Pat. No. 5,118,528, entitled “Process for the preparation of dispersible colloidal systems of a substance in the form of microparticles” and Wendorf et al., WO 2008/051245 A1, entitled “Nanoparticles for use in Immunogenic compositions”. In this technique, for instance, at least one PLGA and at least one cationic surfactant (e.g., selected from those listed above, among others) may be dissolved in one or more organic solvent(s) (e.g., a hydrophilic organic solvent such as acetone, ethanol, DMSO etc. or any mixture thereof). The resulting organic solution may then be combined with a further solvent, which is miscible with the organic solvent while being a non-solvent for the polymer, typically an aqueous solution. The aqueous solution can be, for example, deionized water, normal saline, a buffered solution, such as for example, phosphate-buffered saline (PBS), acetate buffer or a sodium citrate/ethylenediaminetetraacetic acid (sodium citrate/EDTA) or a (4-(2-hydroxyethyl)-1-piperazineethanesulfonic acid) buffer solution. The organic solution and aqueous solution may then be combined in suitable relative volumes, typically from 1:9 to 9:1. For example, the organic solution may be poured, injected dripped into the non-solvent while stirring or homogenizing or shaking, or vice versa. Mixing of the two solutions may also be achieved by standard T/Y-tube mixing techniques, microfluidic mixing, millifluidic mixing, turbulent mixing, trituration mixing or a combination thereof. By selecting a system in which the polymer is soluble in the organic solvent, while being significantly less soluble in the miscible blend of the organic solvent with the non-solvent, a suspension of particles may be formed virtually instantaneously. Subsequently, the organic solvent can be eliminated from the suspension, for example, by evaporation, dialysis or diafiltration.

As previously indicated, in certain embodiments, it is desirable to provide one or more additive (in addition to PLGA), which may be associated with the interior (e.g., entrapped) and/or surface (e.g., by adsorption, covalent attachment, co-lyophilization, etc.) of the particles or may be non-associated with the particles. Such additional species can include, for instance, agents to adjust tonicity or pH, cryoprotective agents, immunological adjuvants, antigens, and so forth.

Such additional species may be provided during the particle formation process. In the above-described particle formation techniques (e.g., w/o/w solvent evaporation, o/w solvent evaporation, nanoprecipitation, etc.), the organic and/or aqueous solutions employed can thus further contain various additives as desired. For example, these additives may be added (α) to an organic solution, if in oil-soluble or oil-dispersible form or (b) to an aqueous solution, if in water-soluble or water-dispersible form.

In some embodiments, one or more additive may be added subsequently to particle formation (typically subsequent to organic solvent removal, as well as subsequent to washing steps or steps in which the particles are dialyzed against water, if any). These additives are frequently added to the particles as an aqueous solution or dispersion. These additives can, for instance, be in solution and/or accumulate at the particle-solution interface, for example, being adsorbed at the particle surface.

Once a suitable composition is formed (e.g., using the above-described or other techniques), it may be lyophilized for future use.

The polynucleotide delivery particles according to the invention can be comprised in oral drug delivery compositions or parenteral, preferably injectable, drug delivery compositions.

Once formulated (and resuspended as necessary), the polynucleotide delivery particles of the invention can be administered parenterally, e.g., by injection (which may be needleless), among other routes of administration. In this regard, the particle compositions are typically supplied lyophilized in a vial or other container which is supplied with a septum or other suitable means for supplying a resuspension medium (e.g., water for injection) and for withdrawing the resultant suspension. A suitable syringe may also be supplied for injection. The compositions can be injected subcutaneously, intradermally, intramuscularly, intravenously, intraarterially, or intraperitoneally, for example.

Other modes of administration include nasal, mucosal, intraoccular, rectal, vaginal, oral and pulmonary administration, and transdermal or transcutaneous applications.

For oral administration the polynucleotide delivery particles can be contained in capsules, preferred capsules are for examples described in WO 2019096833 A1, WO 2020229178 A1, WO 2020229192 A1 and EP Application Serial No. 21175704.2.

In some embodiments, the compositions of the present invention can be used for site-specific targeted delivery. For example, intravenous administration of the compositions can be used for targeting the lung, liver, spleen, blood circulation, or bone marrow. Furthermore, oral administration of the compositions can be used for targeted gastrointestinal tract delivery.

Treatment may be conducted according to a single dose schedule or a multiple dose schedule. A multiple dose schedule is one in which a primary course of administration may be given, for example, with 1-10 separate doses, followed by other doses given at subsequent time intervals, chosen to maintain and/or reinforce the therapeutic response, for example at 1-4 months for a second dose, and if needed, a subsequent dose(s) after several months. The dosage regimen will also be, at least in part, determined by the need of the subject and be dependent on the judgment of the practitioner.

Furthermore, if prevention of disease is desired, the compositions are generally administered prior to the arrival of the primary occurrence of the infection or disorder of interest. If other forms of treatment are desired, e.g., the reduction or elimination of symptoms or recurrences, the compositions are generally administered subsequently to the arrival of the primary occurrence of the infection or disorder of interest.

### EXAMPLES

### Example 1: Testing of DODMA:PLGA for mRNA Encapsulation and Transfection of mRNA into Cells (Comparative Example)

In this example the ionizable lipid DODMA is explored for its capability to work as ionizable surfactant in combination with low molecular weight PLGA. Formation of particles, encapsulation of mRNA and transfection of mRNA into cells is assessed.

### Preparation of DODMA:PLGA Particles

TABLE 1
Materials used for particle preparation.

| Compound | Provider | Catalogue Number |
|---|---|---|
| 1,2-dioleyloxy-3- | NOF America Corporation | COATSOME ® |
| dimethylaminopropane | | CL-E8181DA |
| (DODMA) | | |
| RESOMER ® RG 501 H | Evonik Operations GmbH | n.a. |
| (Poly(D,L-lactide-co- | (Darmstadt, Germany) | |
| glycolide), PLGA) | | |
| CleanCap ® Fluc mRNA | TriLink Bio Technologies | L-7602 |
| | (San Diego, United States) | |

99 μL of ribonucleases free water, 16.5 μL of 100 mM acetate pH 4 buffer and 16.5 μL of firefly luciferase coding mRNA (FLuc mRNA, 1 g/L), altogether forming the aqueous phase, were added into a sterile 1.5 mL safe-lock tube. The aqueous phase was vortexed and spun down. 10.98 μL of a 20 g/L DODMA stock solution in DMSO, 4.39 UL of a 50 g/L RG 501 H stock solution in DMSO and 14.63 μL of DMSO, altogether forming the organic phase, were added into a second 1.5 mL safe-lock tube. In case of DODMA-only particles the respective volume of RESOMER® RG 501 H solution was replaced with DMSO. The organic phase was vortexed (Scientific Industries SI™ Vortex-Genie™ 2) and spun down. 120 μL of aqueous phase from the first tube were taken out and added into the second tube in one shot with a strong pipette burst. The two phases were further mixed by frequent pipetting. The obtained mRNA loaded DODMA:PLGA particles were stored in solution at 4° C. until further use.

### Characterization of DODMA:PLGA Particles

Particle size was measured on a Malvern Zetasizer Nano ZS at an mRNA concentration of 10 ng/μL using water as dispersant.

Gel electrophoresis was conducted with 1 μg of mRNA per well using the Invitrogen™ E-Gel™ Power Snap Electrophoresis System.

Luciferase assay was conducted with human epithelial cells (HeLa). One day before transfection 10,000 cells per well were seeded into a 96-well plate and cultured for 24 h at 37° C. and 5% CO₂. On day 2, old medium was removed and 90 μL of fresh medium was added to the cells. Samples were adjusted to an mRNA concentration of 10 ng/μL using ribonuclease free water for dilution. 10 μL of the respective diluted samples were added to the cells equaling an amount of 100 ng mRNA per well in a total volume of 100 μL. The cells were further incubated for 24 h at 37° C. and 5% CO₂. On day 3, transfection efficiency was determined using a luciferase kit system according to manufacturer's protocol (Promega GmbH). The luminescence signal was quantified by a multiplate reader (Plate reader Infinite® 200 PRO, Tecan).

### Results

TABLE 2
Dynamic light scattering data
for DODMA:PLGA particles

| Particle | z-average [nm] | PDI |
|---|---|---|
| DODMA | 134 | 0.120 |
| DODMA:RG 501 H | 200 | 0.130 |

DODMA as well as a mixture of DODMA and RG 501 H form defined nanoparticles upon mixing of organic phase with aqueous phase containing mRNA. Addition of RG 501 H leads to a significant increase of particle size.

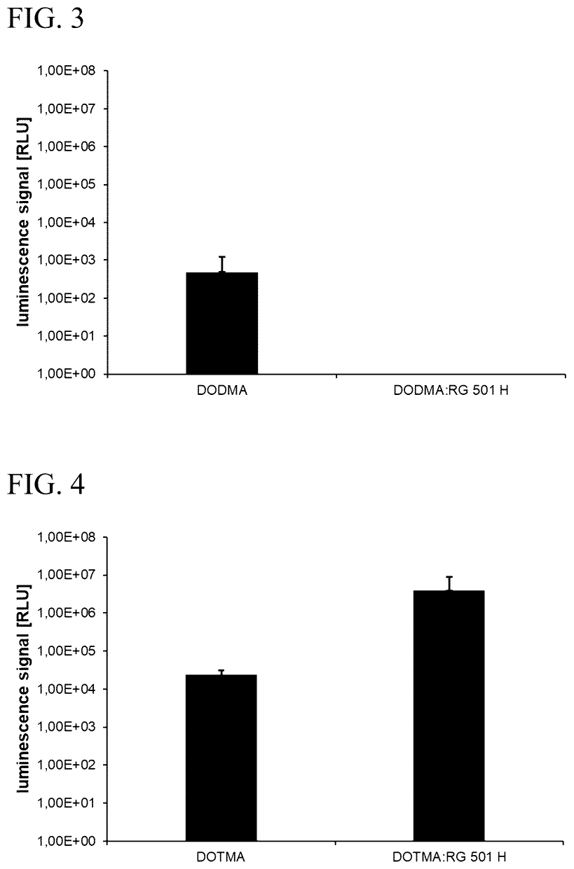

However, according to agarose gel electrophoresis (FIG. 1) the mRNA is not encapsulated into the formed particles, neither in case of DODMA nor in case of the mixture with RG 501 H. Correspondingly, none of the two tested compositions achieve a substantial transfection when incubated with Hela cells (FIG. 3).

The experiment thus clearly demonstrates that a combination of an ionizable surfactant alone with PLGA is insufficient for mRNA encapsulation and transfection.

### Example 2: Testing of DOTMA:PLGA for mRNA Encapsulation and Transfection of mRNA into Cells (Inventive Example)

In this example the cationic lipid DOTMA is explored for its capability to work as ionizable surfactant in combination with low molecular weight PLGA. Formation of particles, encapsulation of mRNA and transfection of mRNA into cells is assessed.

### Preparation of DOTMA:PLGA Particles

TABLE 3
Materials used for particle preparation

| Compound | Provider | Catalogue Number |
|---|---|---|
| 1,2-di-O-octadecenyl-3- | NOF Europe GmbH | COATSOME ® |
| trimethylammonium propane | | CL-E8181TA |
| (chloride salt) (DOTMA) | | |
| RESOMER ® RG 501 H | Evonik Operations GmbH | n.a. |
| (Poly(D,L-lactide-co- | (Darmstadt, Germany) | |
| glycolide), PLGA) | | |
| CleanCap ® Fluc mRNA | TriLink Bio Technologies | L-7602 |
| | (San Diego, United States) | |

66 μL of ribonucleases free water, 11 μL of 100 mM acetate pH 4 buffer and 11 μL of FLuc mRNA (1 g/L), altogether forming the aqueous phase, were added into a sterile 1.5 mL safe-lock tube. The aqueous phase was vortexed and spun down. 7.91 μL of a 20 g/L DOTMA stock solution in DMSO, 6.33 μL of 50 g/L RG 501 H and 5.76 μL of DMSO, altogether forming the organic phase, were added into a second 1.5 mL safe-lock tube. In case of DOTMA-only particles the respective volume of PLGA solution was replaced with DMSO. The organic phase was vortexed and spun down. 80 μL of aqueous phase from the first tube were taken out and added into the second tube in one shot with a strong pipette burst. The two phases were further mixed by frequent pipetting. The obtained mRNA loaded DOTMA:PLGA particles were stored in solution at 4° C. until further use.

### Characterization of DOTMA:PLGA Particles

Particle size was measured on a Malvern Zetasizer Nano ZS at an mRNA concentration of 10 ng/μL using water as dispersant.

Gel electrophoresis was conducted with 1 μg of mRNA per well using the Invitrogen™ E-Gel™ Power Snap Electrophoresis System.

Luciferase assay was conducted with human epithelial cells (HeLa). One day before transfection 10,000 cells per well were seeded into a 96-well plate in DMEM medium and cultured for 24 h at 37° C. and 5% CO₂. On day 2, old medium was removed and 90 μL of fresh medium was added to the cells. Samples were adjusted to an mRNA concentration of 10 ng/μL using RNAse free water for dilution. 10 μL of the respective diluted samples were added to the cells equaling an amount of 100 ng mRNA per well in a total volume of 100 μL. The cells were further incubated for 24 h at 37° C. and 5% CO₂. On day 3, transfection efficiency was determined using a luciferase kit system according to manufacturer's protocol (Promega GmbH). The luminescence signal was quantified by a multiplate reader (Plate reader Infinite® 200 PRO, Tecan).

### Results

TABLE 4
Dynamic light scattering data
for DOTMA:PLGA particles

| Particle | z-average [nm] | PDI |
|---|---|---|
| DOTMA | 104 | 0.340 |
| DOTMA:RG 501 H | 125 | 0.120 |

DOTMA as well as a mixture of DOTMA and RG 501 H form defined nanoparticles upon mixing of organic phase with aqueous phase containing mRNA. In contrast to DODMA, in case of DOTMA particle size is in the same range for all tested mixtures and addition of RG 501 H does not cause a significant size increase. Without being bound to any theory, it is assumed that this can be attributed to the permanent cationic charge of DOTMA which provides a better colloidal stabilization of particles than DODMA.

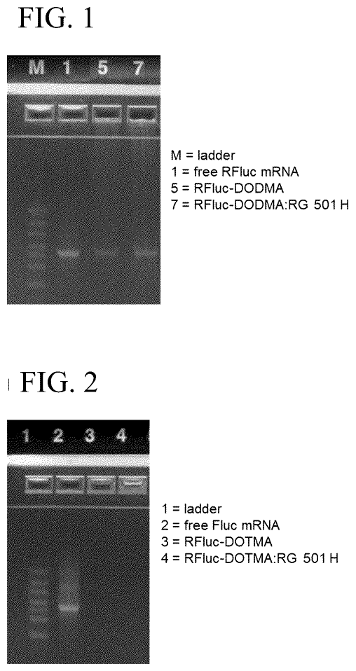

According to agarose gel electrophoresis (FIG. 2) the mRNA is fully encapsulated into the formed particles for all tested compositions. Without being bound to any theory, it is assumed that this again can be attributed by the cationic charge provided to the particles through the use of DOTMA. In case of transfection efficiency (FIG. 4) a significant better performance is observed for the DOTMA:RG 501 H mixed particles compared to DOTMA alone and also compared to all tested previous compositions containing DODMA.

The experiment thus clearly demonstrates that a combination of a cationic surfactant with specific PLGA of the present invention is well suited for mRNA encapsulation and transfection. Moreover, results prove that the specific PLGA of the present invention has a beneficial effect on efficiency of particles described in this invention when it is combined with a cationic surfactant.

### Example 3: Comparison of Low and Mid Molecular Weight PLGA for mRNA Encapsulation and Transfection of mRNA into Cells (Inventive Example)

In this example two PLGA polymers of low and mid molecular weight are applied in combination with DOTMA to encapsulate FLuc mRNA and to form particles. Transfection efficiency and kinetics of the particles in dependence of the applied PLGA is assessed.

### Preparation of DOTMA:PLGA Particles

TABLE 5
Materials used for particle preparation.

| Compound | Provider | Catalogue Number |
|---|---|---|
| 1,2-di-O-octadecenyl-3- | NOF Europe GmbH | COATSOME ® |
| trimethylammonium propane | | CL-E8181TA |
| (chloride salt) (DOTMA) | | |
| RESOMER ® RG 501 H | Evonik Operations GmbH | n.a. |
| (Poly(D,L-lactide-co- | (Darmstadt, Germany) | |
| glycolide)) | | |
| RESOMER® RG 503 H | Evonik Operations GmbH | n.a. |
| (Poly(D,L-lactide-co- | (Darmstadt, Germany) | |
| glycolide)) | | |
| CleanCap ® Fluc mRNA | TriLink BioTechnologies | L-7602 |
| | (San Diego, United States) | |

75.17 UL of ribonucleases free water, 5.5 μL of 200 mM HEPES pH 7 buffer and 11 UL of FLuc mRNA (1 g/L), altogether forming the aqueous phase, were added into a sterile 1.5 mL safe-lock tube. The solution was vortexed and spun down. 7.91 μL of a 20 g/L DOTMA stock solution in DMSO, 6.33 μL of either 50 g/L RG 501 H or 50 g/L RG 503 H stock solutions in DMSO and 2.42 μL of DMSO, altogether forming the organic phase, were added into a second 1.5 mL safe-lock tube. The solution was vortexed and spun down. 83.33 μL of aqueous phase from the first tube were taken out and added into the second tube in one shot with a strong pipette burst. The two phases were further mixed by frequent pipetting. The obtained mRNA loaded DOTMA:PLGA particles were stored in solution at 4° C. until further use.

### Characterization of DOTMA:PLGA Particles

Particle size was measured on a Malvern Zetasizer Nano ZS at an mRNA concentration of 10 ng/μL using water as dispersant.

Luciferase assay was conducted with human epithelial cells (HeLa). One day before transfection 10,000 cells per well were seeded into a 96-well plate in DMEM medium and cultured for 24 h at 37° C. and 5% CO₂. On day 2, old medium was removed and 90 μL of fresh medium was added to the cells. Samples were adjusted to an mRNA concentration of 10 ng/μL using ribonucleases free water for dilution. 10 μL of the respective diluted samples were added to the cells equaling an amount of 100 ng mRNA per well in a total volume of 100 μL. The cells were further incubated for 24 h at 37° C. and 5% CO₂. On day 3, transfection efficiency was determined using a luciferase kit system according to manufacturer's protocol (Promega GmbH). The luminescence signal was quantified by a multiplate reader (Plate reader Infinite® 200 PRO, Tecan).

### Results

TABLE 6
Dynamic light scattering data
for DOTMA:PLGA particles

| Particle | z-average [nm] | PDI |
|---|---|---|
| DOTMA:RG 501 H | 126 | 0.137 |
| (inventive example) | | |
| DOTMA:RG 503 H | 141 | 0.144 |
| (comparative example) | | |

Mixtures of DOTMA with the low molecular weight RESOMER® RG 501 H as well as with the mid molecular weight RESOMER® RG 503 H form defined nanoparticles upon mixing of organic phase with aqueous phase containing mRNA. However, particles as obtained with RG 501 H are larger than those obtained with RG 501 H.

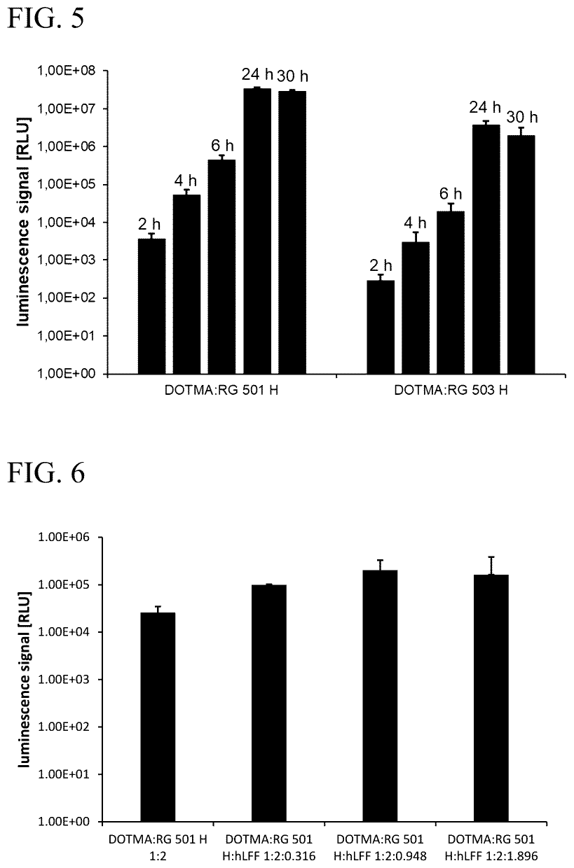

In vitro transfection efficiency of either RG 501 H or RG 503 H containing particles was assessed by luciferase assay at different timepoints of cell incubation (FIG. 5). The luciferase assay demonstrates the functionality of the DOTMA:PLGA particles in transfecting the cells with the encapsulated FLuc mRNA. Importantly, particles comprising RG 501 H perform better than particles with RG 503 H at all tested timepoints. Without being bound to any theory, it is assumed that this can be attributed to the lower molecular weight of RG 501 H as compared to RG 503 H which facilitates particle disassembly and release of the encapsulated mRNA. Ultimately, facilitated mRNA release results in faster transfection kinetics as well as increased overall transfection efficiency of the corresponding particles. Efficiency of the particles described in this invention is thus directly correlated with the PLGA used within the composition.

### Example 4: Coating of DOTMA:PLGA Particles with Human Lactoferrin Fragment to Improve Transfection of mRNA into Intestinal Epithelial Cells (Inventive Example)

In this example DOTMA:RG 501 H particles are coated with the cell-penetrating peptide human lactoferrin fragment (hLFF) in order to further improve uptake and transfection efficiency in intestinal epithelial cells.

Preparation of DOTMA:RG 501 H:hLFF Particles

TABLE 7
Materials used for particle preparation.

| Compound | Provider | Catalogue Number |
|---|---|---|
| 1,2-di-O-octadecenyl-3- | NOF Europe GmbH | COATSOME ® |
| trimethylammonium propane | | CL-E8181TA |
| (chloride salt) (DOTMA) | | |
| RESOMER ® RG 501 H | Evonik Operations GmbH | n.a. |
| (Poly(D,L-lactide-co- | (Darmstadt, Germany) | |
| glycolide)) | | |
| Human lactoferrin fragment | Evonik Operations GmbH | n.a. |
| (hLFF) | (Darmstadt, Germany) | |
| CleanCap ® Fluc mRNA | TriLink Bio Technologies | L-7602 |
| | (San Diego, United States) | |

264 μL of ribonucleases free water, 44 μL of 100 mM acetate pH 4 buffer and 44 μL of FLuc mRNA (1 g/L), altogether forming the aqueous phase, were added into a sterile 1.5 mL safe-lock tube. The aqueous phase was vortexed and spun down. 31.65 μL of a 20 g/L DOTMA stock solution in DMSO, 25.32 μL of a 50 g/L RG 501 H stock solution and 23.03 μL of DMSO, altogether forming the organic phase, were added into a second 1.5 mL safe-lock tube. The organic phase was vortexed and spun down. 320 μL of aqueous phase from the first tube were taken out and added into the second tube in one shot with a strong pipette burst. The two phases were further mixed by frequent pipetting. The obtained mRNA loaded DOTMA:RG 501 H particles were stored in solution at 4° C. until further use. In separate 1.5 mL tubes 2 μL, 6 UL or 12 μL of a 2.5 g/L hLFF stock solution in water were mixed with 48 μL, 44 μL or 38 μL of ribonucleases free water, respectively, so that each tube contained a final volume of 50 μL of diluted hLFF solution. An additional tube contained 50 μL of plain water as negative control. 50 μL of the preformed DOTMA:RG 501 H particle solution were added into each of the prepared tubes, respectively, in one shot with a strong pipette burst followed by frequent pipetting. The coated particles were stored in solution at 4° C. until further use.

Characterization of DOTMA:PLGA:hLFF Particles

Particle size was measured on a Malvern Zetasizer Nano ZS at an mRNA concentration of 10 ng/μL using water as dispersant.

Luciferase assay was conducted with human colorectal adenocarcinoma cells (Caco-2). One day before transfection 10,000 cells per well were seeded into a 96-well plate in DMEM medium and cultured for 24 h at 37° C. and 5% CO₂. On day 2, old medium was removed and 90 μL of fresh medium was added to the cells. Samples were adjusted to an mRNA concentration of 10 ng/μL using RNAse free water for dilution. 10 μL of the respective diluted samples were added to the cells equaling an amount of 100 ng mRNA per well in a total volume of 100 μL. The cells were further incubated for 24 h at 37° C. and 5% CO₂. On day 3, transfection efficiency was determined using a luciferase kit system according to manufacturer's protocol (Promega GmbH). The luminescence signal was quantified by a multiplate reader (Plate reader Infinite® 200 PRO, Tecan).

### Results

TABLE 8
Dynamic light scattering data
for DOTMA:RG 501 H:hLFF particles

| Particle | z-average [nm] | PDI |
|---|---|---|
| DOTMA:RG 501 H, 1:2 | 125 | 0.270 |
| DOTMA:RG 501 H:hLFF, 1:2:0.316 | 114 | 0.250 |
| DOTMA:RG 501 H:hLFF, 1:2:0.948 | 102 | 0.160 |
| DOTMA:RG 501 H:hLFF, 1:2:1.896 | 103 | 0.150 |

The mixture of DOTMA and RG 501 H forms defined nanoparticles upon mixing of organic phase with aqueous phase containing mRNA. Coating of these particles with hLFF causes a slight decrease of particle size.

In case of transfection efficiency in Caco-2 cells (FIG. 6) a significant better performance is observed for the hLFF coated particles when compared to non-coated particles. Maximum of transfection efficiency is reached at a DOTMA:hLFF ratio of 1:0.948. Without being bound to any theory, it is assumed that the positive effect of hLFF stems from improved cellular uptake mediated by hLFF, an effect also known from other cell-penetrating peptides.

The experiment thus clearly demonstrates that DOTMA:PLGA particles can be further improved through coating with cell-penetrating peptides such as hLFF.

### Example 5: Filling of Enteric Coating Capsules with RNA-Containing DOTMA:PLGA Particles and pH Dependent Release of Particles

In this example mRNA containing DOTMA:RG 501 H particles are applied as a relevant model drug product for combination with enteric coating capsules.

### Preparation and Characterization of DOTMA:PLGA Particles

TABLE 9
Materials used for particle preparation.

| Compound | Provider | Catalogue Number |
|---|---|---|
| 1,2-di-O-octadecenyl-3- | NOF Europe GmbH | COATSOME ® |
| trimethylammonium propane | | CL-E8181TA |
| (chloride salt) (DOTMA) | | |
| RESOMER ® RG 501 H | Evonik Operations GmbH | n.a. |
| (Poly(D,L-lactide-co- | (Darmstadt, Germany) | |
| glycolide)) | | |
| CleanCap ® Fluc mRNA | TriLink Bio Technologies | L-7602 |
| | (San Diego, United States) | |

0.5 mL of an DMSO solution containing 9.5 g/L DOTMA and 19.0 g/L RG 501 H (organic phase) were mixed with 2.5 mL of an RNAse free 12 mM HEPES pH 7 solution containing 0.12 g/L FLuc mRNA (aqueous phase) using the Nanoassemblr® Benchtop (PNI) platform. The resulting particle solution was dialyzed (Slide-A-Lyzer™, 10K MWCO) against 10 mM HEPES pH 7 buffer for 3 hours (3× buffer exchange). After dialysis, RNAse free trehalose solution (20 wt %) was added to the particle solution to achieve a final trehalose concentration of 10 wt %. Particles were lyophilized over 48 hours and stored at 4° C. until further use.

Filling of Particles into Capsules

Lyophilized particles were filled into enteric coating capsules (types P0001/21 and 22274/27 disclosed in EP Application Serial No. 21175704.2 in examples 5 and 8) at an amount equal to 100 μg of mRNA per capsule. The filled capsules were sealed and stored at 4° C. until further use.

### Capsule Dissolution Assay

To simulate the gastric environment in fed state the capsules were incubated on a rocking shaker for 2 hours at 37° C. in 10 mL of 0.1 N HCl containing 2 g/L pepsin. Samples for release analysis were taken after 60 and 120 minutes. Subsequently, acidic medium was exchanged against 10 ml of phosphate buffer (18.8 mM phosphate, 145.4 mM NaCl, pH 6.8) and capsules were incubated for another 60 minutes with sample-taking in 15 minutes intervals.

As a negative control pure DOTMA:RG 501 H particles without capsule protection were incubated under the same conditions: 40 μL of particle solution (containing 50 ng/ML mRNA) were mixed with 100 μL 0.1 N HCl containing 2 g/L pepsin and incubated for 2 hours on an orbital shaker at 37° C. and 300 rpm. Afterwards, 60 μL of phosphate buffer were added to the mixture and incubation was continued for another 60 minutes.

After the dissolution assay the media containing the dissolved capsules and PLGA particles were immediately used for the cell transfection assay without intermediate storage. The samples taken at fixed time intervals were stored at 4° C. until further analysis in Ribogreen assay.

### Ribogreen Assay

Ribogreen assay was applied in order to detect and quantify RNA after release of particles from capsules. mRNA concentration was measured at different time intervals to establish release kinetics. The Quant-iT™ RiboGreen™ RNA Assay Kit was used for this assay. As Ribogreen assay is based on measuring fluorescence, black 96-well assay plates with a clear bottom were applied.

The procedure was performed according to manufacturer's protocol with slight adjustments. In a first step, 1×TRIS/EDTA (TE) buffer was prepared by dilution of buffer stock with ribonucleases free water. Particle samples were diluted to a theoretical concentration of 1 μg/ml using TE buffer and added to the plate at a volume of 50 μl. 50 μl of TE buffer were added to the samples in order to measure concentration of accessible mRNA. A calibration standard with the corresponding Fluc mRNA and buffers was applied and added to the same plate as the samples. Working solution of Ribogreen dye was prepared by a 1:100 dilution of reagent with TE-buffer. 100 μl working solution were added to each well followed by thorough mixing through pipetting up and down. Fluorescence signals were measured with a microplate reader at an excitation/emission value of 480/520 nm. All samples and standards were measured in duplicates.

### Luciferase Assay

One day before transfection 10,000 cells per well were seeded into a 96-well plate and cultured for 24 h at 37° C. and 5% CO₂. On day 2, old medium was removed and 90 μL of fresh medium was added to the cells. All samples were adjusted to an mRNA concentration of 10 ng/μL using RNAse free water for dilution. 10 μL of the respective diluted samples were added to the cells equaling an amount of 100 ng mRNA per well in a total volume of 100 μL. The cells were further incubated for 24 h at 37° C. and 5% CO₂. On day 3, transfection efficiency was determined using a luciferase kit system according to manufacturer's protocol (Promega GmbH). By adding a luciferase substrate to the cells, a luminescence signal is generated which can be quantified by a multiplate reader (Plate reader Infinite® 200 PRO, Tecan).

### Results

TABLE 10
DLS data for DOTMA:RG 501 H
particles after different processing steps.

| Particle | z-average [nm] | PDI |
|---|---|---|
| DOTMA:RG 501 H, after preparation | 52.9 | 0.315 |
| DOTMA:RG 501 H, 3 weeks at 4° C. | 50.9 | 0.256 |
| DOTMA:RG 501 H, 3 weeks at −20° C. | 58.7 | 0.193 |
| DOTMA:RG 501 H, after lyophilization | 58.9 | 0.386 |

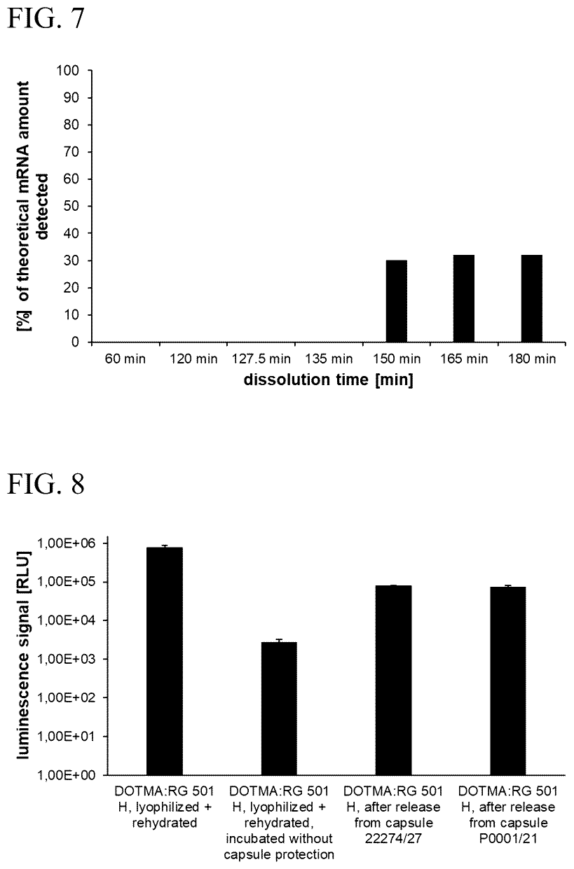

The DLS data demonstrates that defined DOTMA:RG 501 H particles containing mRNA can be produced by a microfluidic method (Nanoassemblr® platform). Compared to samples produced by pipetting the particles obtained from microfluidic mixing are significantly smaller in size. Moreover, particle size stays constant over various processing steps and different storage conditions. The Ribogreen Assay (FIG. 7) clearly proofs a pH dependent release of DOTMA:RG 501 H particles out of the enteric coating capsules, as measured by the signal stemming from accessible mRNA within the particles.

Within 30 minutes after exchange of incubation medium from acidic pH to pH 6.8 particles were fully rehydrated and released from the capsules which went along with complete capsule dissolution. Importantly, no release of particles and mRNA was observed during the 120 minutes incubation in 0.1 N HCl which confirms the structural integrity of the capsules under acidic conditions.

Luciferase transfection assay in human epithelial cells (HeLa) cells was applied in order to assess particle functionality after release from capsules (FIG. 8). Lyophilized particles which were rehydrated only or additionally incubated in fed state simulated gastric and intestinal fluids without capsule protection served as positive and negative controls, respectively. The luciferase assay demonstrates the functionality of the DOTMA:RG 501 H particles after release from capsules as Hela cells incubated with these samples showed distinct expression of the embedded Fluc mRNA. Protection of the particles against fed state simulated gastric and intestinal fluids is further verified by considering the particle negative control which was exposed to the same media without any capsule protection. Transfection efficiency of capsule protected particles is ˜ 2 logs higher than efficiency of non-protected particles confirming a clear beneficial effect of enteric coated capsules on particle functionality. Compared to the positive control, i.e., lyophilized particles rehydrated and directly applied for transfection assay, efficiency of released particles is ˜1 log lower. Without being bound to any theory this could be attributed to dissolved capsule ingredients which might interact with the particles and compromise their integrity. The efficiency drop is comparable for both tested capsule types.

1. A polynucleotide delivery particle, comprising or consisting of
a) at least one poly(lactic-co-glycolide);
b) at least one cationic surfactant; and
c) at least one polynucleotide;
wherein the poly(lactic-co-glycolide) has a weight average molecular weight Mw of 1000 to 9500 g/mol measured via gel permeation chromatography using polystyrene standards and chloroform.

2. The polynucleotide delivery particle according to claim 1, wherein the at least one poly(lactic-co-glycolide) has:
i) a number average molecular weight Mn of 1000 to 3000 g/mol measured via gel permeation chromatography using polystyrene standards and chloroform; and/or
ii) a lactide to glycolide molar ratio ranging from 40:60 to 60:40; and/or
iii) an inherent viscosity of 0.05 to 0.25 dl/g measured via viscometry; and/or
iv) an acid number of 20 to 30 mg KOH/g.

3. The polynucleotide delivery particle according to claim 1, wherein the at least one cationic surfactant is
i) selected from the group consisting of salts of 1,2-di-O-octadecenyl-3-trimethylammonium propane, 1,2-dioleoyl-3-trimethylammonium-propane, N1-[2-((1S)-1-[(3-aminopropyl)amino]-4-[di(3-amino-propyl)amino]butylcarboxamido)ethyl]-3,4-di[oleyloxy]-benzamide, N4-cholesteryl-spermine, 3ß-[N—(N′,N′-dimethylaminoethane)-carbamoyl]cholesterol, O,O′-ditetradecanoyl-N-(α-trimethylammonioacetyl) diethanolamine, 1,2-dilauroyl-sn-glycero-3-ethylphosphocholine, 1,2-dimyristoyl-sn-glycero-3-ethylphosphocholine, 1,2-dipalmitoyl-sn-glycero-3-ethylphosphocholine, 1,2-distearoyl-sn-glycero-3-ethylphosphocholine, 1,2-dioleoyl-sn-glycero-3-ethylphosphocholine, 1-palmitoyl-2-oleoyl-sn-glycero-3-ethylphosphocholine, 1,2-dimyristoleoyl-sn-glycero-3-ethylphosphocholine, dimethyldioctadecylammonium, 1,2-dimyristoyl-3-trimethylammonium-propane, 1,2-dipalmitoyl-3-trimethylammonium-propane, 1,2-stearoyl-3-trimethylammonium-propane, and N-(4-carboxybenzyl)-N,N-dimethyl-2,3-bis(oleoyloxy)propan-1-aminium and 3ß-[N—(N′,N′-dimethylaminoethane)-carbamoyl]cholesterol; or
ii) a 1,2-di-O-octadecenyl-3-trimethylammonium propane salt.

4. The polynucleotide delivery particle according to claim 1, wherein the at least one polynucleotide is a single-stranded polynucleotide or a multi-stranded polynucleotide.

5. The polynucleotide delivery particle according to claim 1, wherein
i) a N/P ratio of the at least one cationic surfactant to the at least one polynucleotide ranges from 1:1 to 50:1; and/or
ii) a molar ratio of the at least one poly(lactic-co-glycolide) to the at least one polynucleotide ranges from 1:1 to 200:1.

6. The polynucleotide delivery particle according to claim 1, further comprising at least one additive selected from the group consisting of buffers; cryoprotective agents; ionizable surfactants; non-ionic surfactants; lipids, and lipids linked to a hydrophilic polymer.

7. The polynucleotide delivery particle according to claim 1, wherein the polynucleotide delivery particle has:
i) a z-average particle size of 1 to 1000 nm measured via dynamic light scattering; and/or
ii) a polydispersity index of 0.01 to 0.5 measured via dynamic light scattering.

8. The polynucleotide delivery particle according to claim 1, wherein the polynucleotide delivery particle has an outer coating layer or at least one additive adsorbed to a surface of the polynucleotide delivery particle.

9. The polynucleotide delivery particle according to claim 8, wherein the outer coating layer comprises a human lactoferrin protein or a fragment thereof or the at least one additive adsorbed to the surface of the polynucleotide delivery particle is a human lactoferrin protein or a fragment thereof.

10. A method of forming the polynucleotide delivery particle according to claim 1, wherein the polynucleotide delivery particle is formed by a nanoprecipitation or a nanoemulsion method.

11. An oral drug delivery composition comprising at least one polynucleotide delivery particle according to claim 1.

12. A parenteral drug delivery composition comprising at least one polynucleotide delivery particle according to claim 1.

13. A medicament, comprising:
the oral drug delivery composition according to claim 11.

14. The polynucleotide delivery particle according to claim 1, further comprising:
d) at least one additive.

15. The polynucleotide delivery particle according to claim 3, wherein the at least one cationic surfactant is a 1,2-di-O-octadecenyl-3-trimethylammonium propane chloride salt.

16. A medicament, comprising:
the parenteral drug delivery composition according to claim 12.
